(19) United States
(12) Patent Application Publication
(10) Pub. No.: US 2008/0228079 A1
(43) Pub. Date: Sep. 18, 2008

(54) CLINICAL UTILIZATION OF CONTRAST AGENTS TO DEFINE SPECIFIC AREAS WITHIN THE MYOCARDIAL WALL TO PROVIDE GUIDANCE AND LOCALIZATION FOR ABLATION, CYROABLATION, OR OTHER TECHNIQUES IN PATIENTS WITH POST MYOCARDIAL INFARCTION

(76) Inventors: Brenda L. Donaldson, Harrison Township, MI (US); Thaddeus H. Chodakauskas, Norco, CA (US); Sachin Vadodaria, Cedar Park, TX (US)

Correspondence Address: MCANDREWS HELD & MALLOY, LTD, 500 WEST MADISON STREET, SUITE 3400, CHICAGO, IL, 60661, US

(21) Appl. No.: 11/687,377

(22) Filed: Mar. 16, 2007

(51) Int. Cl. A61B 8/00; A61B 18/18

(52) U.S. Cl. 600/458; 606/41

(57) ABSTRACT

Certain embodiments of the present invention provide systems and methods for ablating non-viable cardiac tissue. In an embodiment, the method may include locating non-viable cardiac tissue utilizing an electronic medical image. The method may also include locating non-viable cardiac tissue utilizing an ultrasound unit, wherein a contrast agent is used to characterize the non-viable cardiac tissue. The method may also include guiding a catheter to the location of the non-viable cardiac tissue according to the imaging of the ultrasound unit. The method may also include ablating the non-viable cardiac tissue. The ablating of the non-viable cardiac tissue may include cauterizing the non-viable cardiac tissue, cyroablating the non-viable cardiac tissue, or include radiofrequency ablation. The method may also include attempting to locate the non-viable cardiac tissue utilizing an electronic medical image after ablating the non-viable cardiac tissue.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The present invention generally relates to a method for using contrast agents to define specific areas within the myocardial wall. Specifically, the present invention relates using contrast agents to define specific areas within the myocardial wall to provide guidance for ablation, cyroablation, or other techniques or procedures.

Medical patients with impaired left ventricular function are at high risk of sudden death from ventricular arrhythmias. Non-congenital ventricular arrhythmias are most often caused by an area of living myocardial tissue almost completely surrounded by non-viable cardiac tissue. Cardiac tissue has the unique property of being able to self-stimulate. Tissue areas surrounded by non-viable cardiac tissue do not receive the stimulus from the surrounding tissues. Therefore, the cardiac tissue that is surrounded by non-viable cardiac tissue may “fire” on its own. When this stimulus leaves the area surrounding the non-viable cardiac tissue and occurs at a critical time when the heart is susceptible to arrhythmias, ventricular tachycardia can be propagated.

Medical patients with coronary artery disease with prior myocardial infarction may experience sudden cardiac death during the first year due to ventricular tachycardia or fibrillation. The mechanism of ventricular tachycardia in most of these patients is myocardial reentry. Impaired conduction due to myocardial scarring for reentry is may be based on tachyarrhythmias, triggered activity, and increased normal or abnormal automaticity, which cause rhythm disturbances with patients who have coronary artery disease.

The ICD (Implanted Cardiac Defibrillator) is known to be effective in treating ventricular arrhythmias by, for example, monitoring the cardiac rate and delivering therapy when the rate exceeds the programmed rate cutoff. There have been clinical trials involving ICD's which show clinical benefits and improved quality of health. The implantation of an ICD, however, is relatively expensive. There are large numbers of patients with impaired ventricular function, and to implant ICDs in all would place a considerable burden on the health care system.

An alternative technique to treat patients with ventricular arrhythmias is, for example, performing radiofrequency (RF) ablation procedures. Currently, in order to perform an RF ablation procedure, the physician uses a combination of catheters, mapping systems, and x-ray equipment to assist in localizing and guiding the ablation catheter to the location of the surrounding non-viable cardiac tissue in order to interrupt the re-entry point. This RF ablation procedure may be a long procedure for both the physician and patient to endure, and may require a high level of skill by the physician. In addition, the procedure also may expose the patient and the physician to relatively high doses of x-ray.

Accordingly, a need exists to improve the technique to perform a cardiac ablation procedure. Such an improvement may reduce the length of the procedure and reduce the radiation exposure for both the physician and patient. In addition, an improved technique to perform an ablation may allow more accurate localization. A more accurate localization may reduce complications of the procedure and minimize recurrent arrhythmias, thereby reducing costs to the patient.

### SUMMARY OF THE INVENTION

Certain embodiments of the present invention may include a system for locating non-viable cardiac tissue. The system may include an electronic medical imaging unit to acquire electronic medical images of non-viable cardiac tissue. The electronic medical imaging unit may be a Picture Archival Communication System (PACS) unit. The system may also include an ultrasound probe to receive data regarding non-viable cardiac tissue. A contrast agent may be used to characterize the non-viable cardiac tissue. The system may also include an ultrasound unit for receiving data from the ultrasound probe and generating ultrasound images. The system may also include a display unit for displaying the ultrasound images and/or the electronic medical images of the non-viable cardiac tissue. The ultrasound images may be displayed in real-time on the display unit. The electronic medical images of the non-viable cardiac tissue may be translucently overlaid on the ultrasound images.

Certain embodiments of the present invention include a method for ablating non-viable cardiac tissue. The method may include locating non-viable cardiac tissue utilizing an electronic medical image. The method may also include locating non-viable cardiac tissue utilizing an ultrasound unit. A contrast agent may be used to characterize the non-viable cardiac tissue. The method may also include guiding a catheter to the location of the non-viable cardiac tissue in according to the imaging of the ultrasound unit. The method may also include ablating the non-viable cardiac tissue. The non-viable cardiac tissue may be ablated by cauterizing the non-viable cardiac tissue. In addition, the non-viable cardiac tissue may be ablated by cyroablating the non-viable cardiac tissue. In addition, the non-viable cardiac tissue may be ablated by utilizing radiofrequency ablation. The method may further include attempting to locate the non-viable cardiac tissue utilizing an electronic medical image after ablating the non-viable cardiac tissue. The electronic medical image may be acquired using computerized tomography, electron beam tomography, and/or magnetic resonance.

Certain embodiments of the present invention include a method for ablating non-viable cardiac tissue. The method includes locating non-viable cardiac tissue utilizing an ultrasound unit. A contrast agent is used to delineate the non-viable cardiac tissue. The contrast agent may not be absorbed by the non-viable cardiac tissue. The contrast agent may be absorbed by the viable cardiac tissue. The method also includes guiding a catheter to the location of the non-viable cardiac tissue in real-time in according to the imaging of the ultrasound unit. The method also includes ablating the non-viable cardiac tissue. The non-viable cardiac tissue may be ablated by cauterizing the non-viable cardiac tissue. In addition, the non-viable cardiac tissue may be ablated by cyroablating the non-viable cardiac tissue. In addition, the non-viable cardiac tissue may be ablated by utilizing radiofrequency ablation. The method may further include attempting to locate the non-viable cardiac tissue utilizing an electronic medical image after ablating the non-viable cardiac tissue. The electronic medical image may be acquired using computerized tomography, electron beam tomography, and/or magnetic resonance.

### BRIEF DESCRIPTION OF THE DRAWINGS

FIG. 1 illustrates an example of a system for locating non-viable cardiac tissue that may be used in accordance with an embodiment of the present invention.

FIG. 2 illustrates a method for ablating non-viable cardiac tissue in accordance with an embodiment of the present invention.

FIG. 3 illustrates and example of an ultrasound contrast agent in operation.

FIG. 4 illustrates a method for ablating non-viable cardiac tissue in accordance with an embodiment of the present invention.

FIG. 5 illustrates a method for ablating non-viable cardiac tissue in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

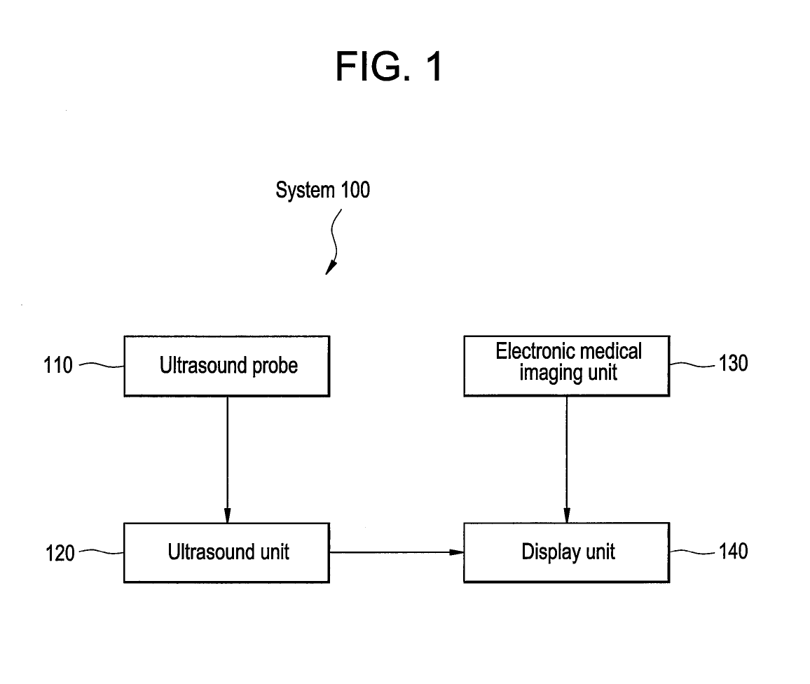

FIG. 1 illustrates a system 100 for locating non-viable cardiac tissue. The system 100 includes an ultrasound probe 110. The ultrasound probe 110 may be equipment or software that receives data and ultrasound images, including data regarding non-viable cardiac tissue. The system 100 also includes an ultrasound unit 120. The ultrasound unit 120 may be equipment or software that processes data and ultrasound images obtained from the ultrasound probe 110. The system 100 also includes an electronic medical imaging unit 130. The electronic medical imaging unit 130 may represent any equipment or software that permits electronic medical images, such as x-rays, ultrasound, CT, MRI, gated MRI, EBT, MR, or nuclear medicine for example, to be electronically acquired, stored, or transmitted for viewing and operation. The electronic medical imaging unit 130 may receive input from a user. The electronic medical imaging unit 130 may be used to acquire electronic medical images of non-viable cardiac tissue. The electronic medical images unit 130 may be connected to other devices as part of an electronic network. The electronic medical imaging unit 130 may be connected to network physically, by a wire, or through a wireless medium.

In an embodiment, the electronic medical images unit 130 may be, or may be part of, a picture archival communication system (PACS). The system 100 also includes a display unit 140. The display unit 140 may be any display device for displaying images, including ultrasound images and the electronic medical images of the non-viable cardiac tissue. In an embodiment, the ultrasound images are displayed in real-time on the display unit. In an embodiment, the display unit 140 may receive data from the ultrasound unit 120 and the electronic medical image unit 140. In an embodiment, the electronic medical images of the non-viable cardiac tissue may be translucently overlaid on the ultrasound images.

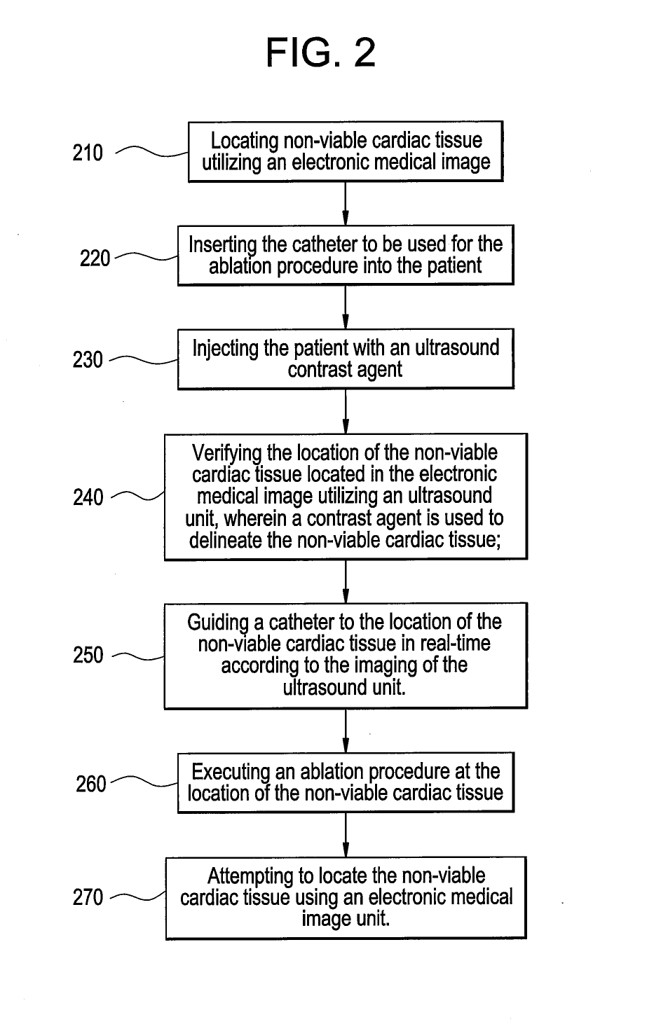

FIG. 2 illustrates a method 200 for ablating non-viable cardiac tissue in accordance with an embodiment of the present invention. At step 210, a physician locates non-viable cardiac tissue utilizing an electronic medical image. The physician may locate the non-viable cardiac tissue utilizing a computerized tomography (CT) unit, an electron beam tomography (EBT) unit, a magnetic resonance (MR) unit, or other imaging technique. The electronic medical image may be acquired before the ablating procedure and used for surgery planning by the physician. In an embodiment, the electronic medical image may be stored as part of the patient's medical profile on a PACS system. In an embodiment, the electronic medical image illustrating the non-viable cardiac tissue is available prior to and during the ablation procedure for viewing by the physician.

After a physician has located the non-viable cardiac tissue using an electronic medical image as in step 210, the physician is generally prepared to begin surgery. Step 210 may occur immediately before the ablating procedure, or hours, days, or even weeks before the ablating procedure. Generally, the first step in the ablating procedure is step 220, where the catheter to be used for the ablation procedure is inserted into the patient. In an embodiment, the catheter may be an ICE catheter.

At step 230, the physician may inject the patient with a contrast agent. The contrast agent may be any ultrasound contrast agent that is capable of delineating the non-viable cardiac tissue. For example, the contrast agent may be Optison or Albunex. Using a contrast agent may help a physician identify an area of non-viable cardiac tissue in relation to the area of viable cardiac tissue.

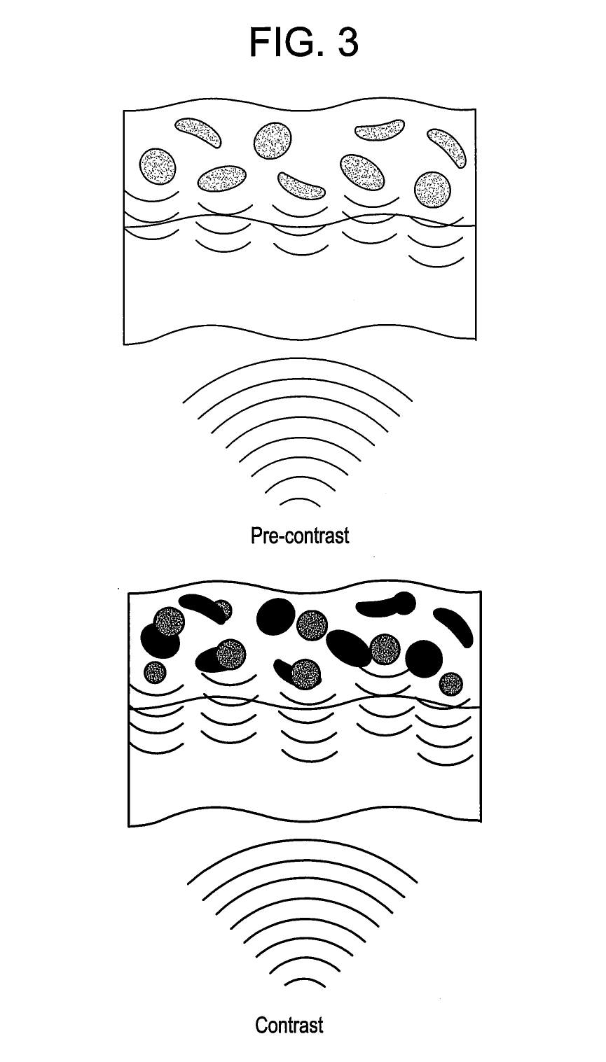

As is shown in FIG. 3, the contrast agent may characterize the non-viable cardiac tissue. The contrast may be illustrated by different types of tissues at different times, including non-viable cardiac tissues. The acoustic impedance of the contrast agent may be lower than that of the blood. As a result, ultrasound waves may be scattered and reflected at the microsphere-blood interface, and return to the ultrasound transducer. Ultimately, the microsphere-blood interface may be visualized in the ultrasound image. The ultrasound contrast agents may provide for increased diagnostic capabilities in a variety of normal and abnormal vessels and organs throughout the body. The ultrasound contrast agents may enhance tumor vascularity, delineate areas of ischemia, as well as improve visualization of vascular stenosis.

At step 240, a physician may locate the non-viable cardiac tissue utilizing an ultrasound unit. The physician may locate the non-viable cardiac tissue by utilizing the contrast agent. In an embodiment, the viable cardiac tissue, which absorbs the contrast agent, may appear brighter on an ultrasound image than non-viable cardiac tissue, which does not absorb the contrast agent, and may appear dark. A physician may compare the location of the non-viable cardiac tissue as identified by the electronic medical images with the location of the non-viable cardiac tissue as identified with the contrast agent on the ultrasound image. In such a manner, the physician may locate the non-viable cardiac tissue using the ultrasound unit in real-time during surgery.

As the contrast agent may delineate the non-viable cardiac tissue, the ultrasound unit may display the location of the non-viable cardiac tissue as well as the real-time location of the catheter in relation to the non-viable cardiac tissue. At step 250, the physician may guide the catheter to the location of the non-viable cardiac tissue in real-time according to the imaging of the ultrasound unit. In an embodiment, the journey from entry point to heart muscle may be navigated by images created by the ultrasound unit, including images of the non-viable cardiac tissue and real-time images of the catheter.

At step 260, once the catheter has reached the location of the non-viable cardiac tissue an ablation may be performed. In an embodiment, energy may be used to destroy a small amount of tissue, ending the disturbance of electrical flow through the heart and restoring a healthy heart rhythm. In an embodiment, this energy may take the form of radiofrequency energy. In another embodiment, this energy may be used to cauterize the non-viable cardiac tissue. In another embodiment, this energy may take the form of intense cold, which freezes, or cryoablates the tissue. Other energy sources may be utilized.

At step 270, after steps 210-260 have been performed, a physician may attempt to locate the non-viable cardiac tissue using an electronic medical image unit. Step 270 may be executed in a similar manner as step 210 in that a physician may locate the non-viable cardiac tissue utilizing a computerized tomography (CT) unit, an electron beam tomography (EBT) unit, a magnetic resonance (MR) unit, or other imaging technique. At step 270, however, the physician is evaluating the success of the procedure.

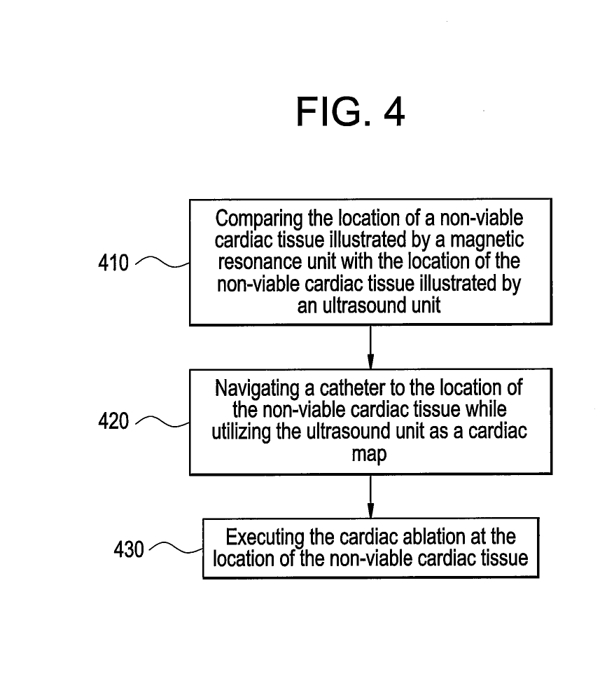

FIG. 4 illustrates a method 400 for performing an ablation procedure in accordance with an embodiment of the present invention. At step 410, a physician may compare the location of a non-viable cardiac tissue as illustrated by an electronic imaging unit, such as for example a computerized tomography (CT) unit, an electron beam tomography (EBT) unit, a magnetic resonance (MR) unit, or other imaging technique, with the location of the non-viable cardiac tissue as illustrated by an ultrasound unit. The ultrasound unit may illustrate the non-viable cardiac tissue by using a contrast agent. In an embodiment, the viable cardiac tissue may appear brighter on an ultrasound image and non-viable cardiac tissue may appear darker. A physician may compare the location of the non-viable cardiac tissue as identified by the electronic medical image with the location of the non-viable cardiac tissue as identified with the contrast agent on the ultrasound image. In such a manner, the physician may locate the non-viable cardiac tissue using the ultrasound unit in real-time.

At step 420, the physician may utilize the ultrasound image to navigate the catheter to the location of the non-viable cardiac tissue. As the contrast agent may identify the non-viable cardiac tissue, the ultrasound unit may display the location of the non-viable cardiac tissue as well as the real-time location of the catheter in relation to the non-viable cardiac tissue.

At step 430, once the catheter has reached the location of the non-viable cardiac tissue, an ablation may be performed. In an embodiment, energy may be used to destroy a small amount of tissue, ending the disturbance of electrical flow through the heart and restoring a healthy heart rhythm. In an embodiment, this energy may take the form of radiofrequency energy. In another embodiment, this energy may be used to cauterize the non-viable cardiac tissue. In another embodiment, this energy may take the form of intense cold, which freezes, or cryoablates the tissue. Other energy sources may be utilized.

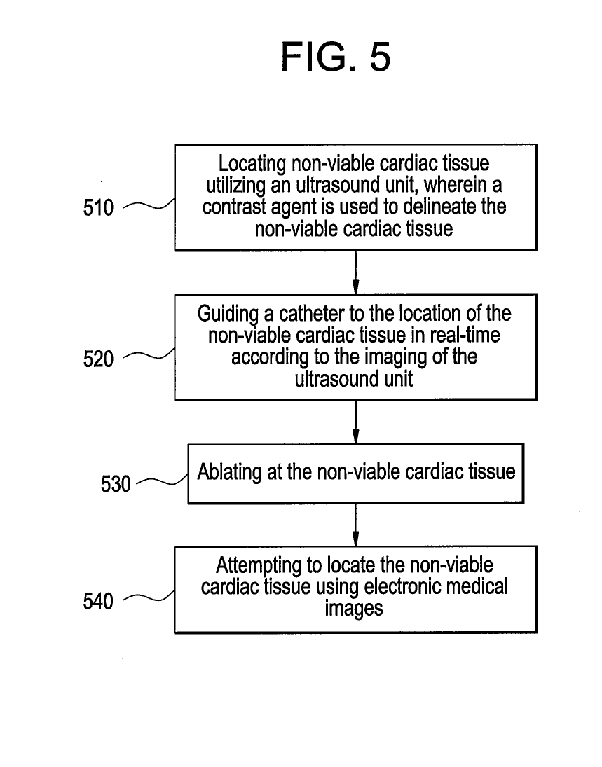

FIG. 5 illustrates a method 500 for ablating non-viable cardiac tissue in accordance with an embodiment of the present invention. At step 510, a physician may locate the position of non-viable cardiac tissue utilizing a contrast agent and an ultrasound unit. The ultrasound unit may illustrate the non-viable cardiac tissue by using a contrast agent. In an embodiment, the viable cardiac tissue may absorb the contrast agent and may appear brighter on an ultrasound image than a non-viable cardiac tissue, which does not absorb the contrast agent, and may appear dark. In such a manner, the physician may locate the non-viable cardiac tissue using the ultrasound unit in real-time.

At step 520, the physician may guide the catheter to the location of the non-viable cardiac tissue in real-time according to the imaging of the ultrasound unit. In an embodiment, the journey from entry point to heart muscle may be navigated by images created by the ultrasound unit, including images of the non-viable cardiac tissue and real-time images of the catheter.

At step 530, once the catheter has reached the location of the non-viable cardiac tissue an ablation may be performed. In an embodiment, energy may be used to destroy a small amount of tissue, ending the disturbance of electrical flow through the heart and restoring a healthy heart rhythm. In an embodiment, this energy may take the form of radiofrequency energy. In another embodiment, this energy may be used to cauterize the non-viable cardiac tissue. In another embodiment, this energy may take the form of intense cold, which freezes, or cryoablates the tissue. Other energy sources may be utilized.

In order to evaluate the success of the procedure, at step 540, a physician may attempt to locate the non-viable cardiac tissue using electronic medical images. A physician may locate the non-viable cardiac tissue utilizing a computerized tomography (CT) unit, an electron beam tomography (EBT) unit, a magnetic resonance (MR) unit, or other imaging technique.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A system for locating non-viable cardiac tissue, said system comprising:
an electronic medical imaging unit to acquire electronic medical images of non-viable cardiac tissue;
an ultrasound probe to receive data regarding non-viable cardiac tissue, wherein a contrast agent is used to characterize the non-viable cardiac tissue;
an ultrasound unit for receiving data from the ultrasound probe and generating ultrasound images; and, a display unit for displaying the ultrasound images.

2. The system of claim 1, wherein the display unit further displays the electronic medical images of the non-viable cardiac tissue.

3. The system of claim 2, wherein the electronic medical images of the non-viable cardiac tissue are translucently overlaid on the ultrasound images.

4. The system of claim 1, wherein the electronic medical imaging unit is a Picture Archival Communication System (PACS) unit.

5. The system of claim 1, wherein the ultrasound images are displayed in real-time on the display unit.

6. A method for ablating non-viable cardiac tissue, said method comprising:
locating non-viable cardiac tissue utilizing an electronic medical image;
locating non-viable cardiac tissue utilizing an ultrasound unit, wherein a contrast agent is used to characterize the non-viable cardiac tissue;
guiding a catheter to the location of the non-viable cardiac tissue according to the imaging of the ultrasound unit; and, ablating the non-viable cardiac tissue.

7. The method of claim 6, wherein said ablating step includes cauterizing the non-viable cardiac tissue.

8. The method of claim 6, wherein said ablating step includes cyroablating the non-viable cardiac tissue.

9. The method of claim 6, wherein said ablating step includes radiofrequency ablation.

10. The method of claim 6, further including attempting to locate the non-viable cardiac tissue utilizing an electronic medical image after ablating the non-viable cardiac tissue.

11. The method of claim 6, wherein the electronic medical image is acquired using computerized tomography.

12. The method of claim 6, wherein the electronic medical image is acquired using electron beam tomography.

13. The method of claim 6, wherein the electronic medical image is acquired using magnetic resonance.

14. A method for ablating non-viable cardiac tissue, said method comprising:
locating non-viable cardiac tissue utilizing an ultrasound unit, wherein a contrast agent is used to delineate the non-viable cardiac tissue;
guiding a catheter to the location of the non-viable cardiac tissue in real-time in according to the imaging of the ultrasound unit; and,
ablating the non-viable cardiac tissue.

15. The method of claim 14, wherein said ablating step includes cauterizing the non-viable cardiac tissue.

16. The method of claim 14, wherein said ablating step includes cyroablating the non-viable cardiac tissue.

17. The method of claim 14, wherein said ablating step includes radiofrequency ablation.

18. The method of claim 14, further including attempting to locate the non-viable cardiac tissue utilizing electronic medical images after ablating the non-viable cardiac tissue.

19. The method of claim 14, wherein said contrast agent is not absorbed by the non-viable cardiac tissue.

20. The method of claim 19, wherein said contrast agent is absorbed by the viable cardiac tissue.
